(19) United States
(12) Patent Application Publication
(10) Pub. No.: US 2024/0238586 A1
(43) Pub. Date: Jul. 18, 2024

(54) ELECTRODE FOR NERVE STIMULATOR COMPRISING BIOCOMPATIBLE SOLID ELECTROLYTE, METHOD FOR MANUFACTURING SAME, AND NERVE STIMULATOR COMPRISING SAME

(71) Applicants: INDUSTRY-UNIVERSITY COOPERATION FOUNDATION HANYANG UNIVERSITY, Seoul (KR); KOREA ADVANCED INSTITUTE OF SCIENCE AND TECHNOLOGY, Daejeon (KR)

(72) Inventors: Do Hwan KIM, Seoul (KR); Steve PARK, Daejeon (KR); Joo Sung KIM, Yongin-si, Gyeonggi-do (KR); Jun Jae PARK, Seoul (KR); Yun Ah KIM, Seoul (KR); Hyuk Min KWEON, Uijeongbu-si, Gyeonggi-do (KR)

(73) Assignees: IUCF-HYU (INDUSTRY-UNIVERSITY COOPERATION FOUNDATION HANYANG UNIVERSITY), Seoul (KR); KOREA ADVANCED INSTITUTE OF SCIENCE AND TECHNOLOGY, Daejeon (KR)

(21) Appl. No.: 18/563,990

(22) PCT Filed: May 24, 2022
(86) PCT No.: PCT/KR2022/007328

§ 371 Date: Nov. 24, 2023

(30) Foreign Application Priority Data
May 24, 2021 (KR) 10-2021-0066411

(51) Int. Cl. A61N 1/05; A61B 5/266

(52) U.S. Cl. CPC A61N 1/0551; A61B 5/266; A61B 2562/0217; A61B 2562/125

(57) ABSTRACT

Disclosed are a nerve stimulator electrode and a method of manufacturing the same. More particularly, the present invention provides a nerve stimulator electrode including biocompatible solid electrolyte; and an ion diffusion barrier formed on the biocompatible solid electrolyte, wherein the biocompatible solid electrolyte includes an ionic liquid and a biocompatible polymer matrix.

### CROSS-REFERENCE TO RELATED APPLICATION

This application is a National Stage Entry of PCT International Application No. PCT/KR2022/007328, which was filed on May 24, 2022, and which claims priority to Korean Patent Application No. 10-2021-0066411, filed on May 24, 2021 in the Korean Intellectual Property Office, the disclosure of which is incorporated herein by reference.

### TECHNICAL FIELD

The present invention relates to a nerve stimulator electrode containing a biocompatible solid electrolyte, a method of manufacturing the same and a nerve stimulator including the same, and more particularly to a biocompatible solid electrolyte-containing nerve stimulator electrode capable of delivering electrical stimulation long-term/continuously when attached to nerves in the body because it is manufactured using a biocompatible solid electrolyte material, a method of manufacturing the same and a nerve stimulator including the same.

### BACKGROUND ART

Nerve electrodes can be used in the field of neural interfaces in vivo or in vitro. More specifically, nerve electrodes can be used to provide electrical stimulation to nerves or to measure or record nerve signals. In particular, a nerve stimulator electrode can be used as a treatment method to treat various neurological diseases by applying electrical stimulation to nerves, organs, and tissues or to improve the regeneration speed of damaged nerves by accelerating cell activation and axon differentiation.

As conventional technologies for conductive materials and material structures that contact with nerve, 1) Michigan probe and Utah array based on a silicon material, 2) a polymer probe based on polyimide or SU-8, 3) an elastomer probe using PDMS substrate (elastomeric probe), 4) an ultrathin array based on a conductive polymer and a gold material, and 5) a freestanding mech probe (freestanding Mound Electrical Calibration Heater probe), and the like have been studied.

However, since the conventional technologies use silicon or metal-based materials with high mechanical modulus, it can easily cause damage and pressure to nerves, causing an immune response. In addition, it is difficult to inject consistent electrical stimulation by causing a decrease and change in the contact area between an electrode and the nerve.

Accordingly, many methods have been studied to make metal materials very thin, form them on flexible polymer substrates, and use conductive polymers. However, since conductive polymer materials deliver and inject charges through oxidation/reduction reactions with ions present in biological tissues and body fluids, the generation of hydrogen ions and hydrogen gas/chlorine gas during the reaction may form a gap between an electrode and the nerve and put pressure on the nerve, causing various diseases.

Therefore, research is needed on a nerve stimulator electrode that does not cause immune response and physical/chemical damage to living tissue when in contact with nerves.

### DISCLOSURE

### Technical Problem

Therefore, the present invention has been made in view of the above problems, and it is one object of the present invention to provide a biocompatible solid electrolyte-containing nerve stimulator electrode that exhibits similar mechanical properties to living tissue due to application of a biocompatible solid electrolyte material and, thus, does not cause immune response and physical damage to living tissue when in contact with nerves, and that can deliver electrical signals applied from a current signal generator to nerves because it maintains a stable nervous system even when body movement exists; a method of manufacturing the biocompatible solid electrolyte-containing nerve stimulator electrode; and a nerve stimulator including the biocompatible solid electrolyte-containing nerve stimulator electrode.

It is another object of the present invention to provide a nerve stimulator electrode that can be inserted into the body for a long time by suppressing the ion exchange reaction between body fluid and an electrode because an ion diffusion barrier is formed on the surface of the biocompatible solid electrolyte; a method of manufacturing the nerve stimulator electrode; and a nerve stimulator including the nerve stimulator electrode.

It is yet another object of the present invention to provide a nerve stimulator electrode that can prevent side effects caused by electrochemical oxidation/reduction reactions because it is based on a capacitive charge injection mechanism.

### Technical Solution

In accordance with an aspect of the present invention, the above and other objects can be accomplished by the provision of a nerve stimulator electrode, including: a biocompatible solid electrolyte; and an ion diffusion barrier formed on the biocompatible solid electrolyte, wherein the biocompatible solid electrolyte includes an ionic liquid and a biocompatible polymer matrix.

The biocompatible solid electrolyte may allow ion movement and form an electric double layer under an electric field.

At least one of mechanical modulus and tensile yield point of the biocompatible solid electrolyte may be controlled depending upon a content of the ionic liquid.

Mechanical properties of the biocompatible solid electrolyte may be controlled depending upon a crosslinking density of the biocompatible polymer.

The ionic liquid may include a choline cation and a carboxylic anion paired with the choline cation, wherein the choline cation includes at least one of choline bicarbonate, choline hydroxide and choline chloride, and the carboxylic anion includes at least one of acetate, propionate, glycolate, benzoate, tiglate, malate, succinate, tartrate, fumarate and maleate.

The biocompatible polymer may include at least one of chitosan, collagen, gelatin, fucoidan, alginate, hyaluronic acid, cellulose, polylactide, polyglycolide, polycaprolactone, polytrimethylenecarbolinecarbonate, polyaminoacid, polyorthoester, polyethylene oxide and a copolymer thereof.

The ion diffusion barrier may include a first interface functional group and a second interface functional group on a surface thereof, wherein the first interface functional group induces at least one of hydrogen and covalent bonds with the solid electrolyte, and the second interface functional group induces at least one of hydrogen and covalent bonds with nerve.

A nano-micro pattern may be included on a surface of the ion diffusion barrier.

The ion diffusion barrier may include at least one of graphene, graphene oxide, Mxenes, 2D transition metal carbides, 2D transition metal carbides nitrides, transition metal dichalcogenides (TMDCs), 2D metal organic frameworks (MOFs) and 2D covalent organic frameworks (COFs).

In accordance with another aspect of the present invention, there is provided a method of manufacturing a nerve stimulator electrode, the method including: forming an ion diffusion barrier on a substrate; coating a biocompatible solid electrolyte solution including an ionic liquid and a biocompatible polymer on the ion diffusion barrier; curing the coated biocompatible solid electrolyte solution; and separating the cured biocompatible solid electrolyte from the substrate.

The forming may further include first surface-treating of the ion diffusion barrier.

The first surface-treating may include at least one of silane functionalization and dopamine/polydopamine functionalization.

After the separating, second surface-treating of the ion diffusion barrier may be performed.

In accordance with yet another aspect of the present invention, there is provided a nerve stimulator, including: a pulse generator for generating electrical nerve stimulation pulses; the nerve stimulator electrode according to claim 1 that is in contact with a nerve; and a wire connection for electrically connecting the pulse generator and the nerve stimulator electrode.

### Advantageous Effects

In accordance with an embodiment of the present invention, provided are a nerve stimulator electrode that exhibits similar mechanical properties to living tissue due to application of a biocompatible solid electrolyte material and, thus, does not cause immune response and physical damage to living tissue when in contact with nerves, and that can deliver electrical signals applied from a current signal generator to nerves because it maintains a stable nervous system even when body movement exists; a method of manufacturing the nerve stimulator electrode; and a nerve stimulator including the nerve stimulator electrode.

In accordance with an embodiment of the present invention, provided are a nerve stimulator electrode that can be inserted into the body for a long time by suppressing the ion exchange reaction between body fluid and an electrode because an ion diffusion barrier is formed on the surface of the biocompatible solid electrolyte; a method of manufacturing the nerve stimulator electrode; and a nerve stimulator including the nerve stimulator electrode.

In accordance with an embodiment of the present invention, provided are a nerve stimulator electrode that can prevent side effects caused by electrochemical oxidation/reduction reactions because it is based on a capacitive charge injection mechanism.

### DESCRIPTION OF DRAWINGS

FIG. 1 illustrates a sectional view of a nerve stimulator electrode according to an embodiment of the present invention.

FIG. 2 is a schematic diagram illustrating a method of manufacturing the nerve stimulator electrode according to an embodiment of the present invention.

FIG. 3 illustrates a synthesis process of an ionic liquid used in the nerve stimulator electrode according to an embodiment of the present invention.

FIG. 4 illustrates the cross-linking reaction of a biocompatible polymer used in the nerve stimulator electrode according to an embodiment of the invention.

FIG. 5 illustrates a schematic diagram of a first surface treatment step of an ion diffusion barrier in the method of manufacturing the nerve stimulator electrode according to an embodiment of the present invention.

FIG. 6 illustrates a schematic diagram of a second surface treatment step of an ion diffusion barrier in the method of manufacturing the nerve stimulator electrode according to an embodiment of the present invention.

FIG. 7 is a graph illustrating the mechanical properties of the nerve stimulator electrode according to an embodiment of the present invention dependent upon the content of an ionic liquid.

FIG. 8 is a graph illustrating changes in electrochemical properties of the nerve stimulator electrode according to an embodiment of the present invention dependent upon the content of an ionic liquid.

FIG. 9 illustrates a charge injection amount dependent upon the number of layers of an ion diffusion barrier of the nerve stimulator electrode according to an embodiment of the present invention.

FIG. 10 illustrates a graph showing changes in electrochemical properties of the nerve stimulator electrode according to an embodiment of the present invention including an ion diffusion barrier and an image showing the transmittance within an electrode of a PBS buffer solution.

FIG. 11 illustrates an image and graph showing the ion outflow effect dependent upon the physical tension of the nerve stimulator electrode according to an embodiment of the present invention including an ion diffusion barrier.

FIG. 12 is a graph illustrating the charge injection efficiency of the nerve stimulator electrode according to an embodiment of the present invention.

### BEST MODE

The present invention will now be described more fully with reference to the accompanying drawings and contents disclosed in the drawings. However, the present invention should not be construed as limited to the exemplary embodiments described herein.

The terms used in the present specification are used to explain a specific exemplary embodiment and not to limit the present inventive concept. Thus, the expression of singularity in the present specification includes the expression of plurality unless clearly specified otherwise in context. It will be further understood that the terms “comprise” and/or “comprising”, when used in this specification, specify the presence of stated components, steps, operations, and/or elements, but do not preclude the presence or addition of one or more other components, steps, operations, and/or elements thereof.

It should not be understood that arbitrary aspects or designs disclosed in “embodiments”, “examples”, “aspects”, etc. used in the specification are more satisfactory or advantageous than other aspects or designs.

In addition, the expression “or” means “inclusive or” rather than “exclusive or”. That is, unless otherwise mentioned or clearly inferred from context, the expression “x uses a or b” means any one of natural inclusive permutations.

In addition, as used in the description of the invention and the appended claims, the singular forms “a”, “an” and “the” are intended to include the plural forms as well, unless context clearly indicates otherwise.

Although terms used in the specification are selected from terms generally used in related technical fields, other terms may be used according to technical development and/or due to change, practices, priorities of technicians, etc. Therefore, it should not be understood that terms used below limit the technical spirit of the present invention, and it should be understood that the terms are exemplified to describe embodiments of the present invention.

Also, some of the terms used herein may be arbitrarily chosen by the present applicant. In this case, these terms are defined in detail below. Accordingly, the specific terms used herein should be understood based on the unique meanings thereof and the whole context of the present invention.

Meanwhile, terms such as “first” and “second” are used herein merely to describe a variety of constituent elements, but the constituent elements are not limited by the terms. The terms are used only for the purpose of distinguishing one constituent element from another constituent element.

In addition, when an element such as a layer, a film, a region, and a constituent is referred to as being “on” another element, the element can be directly on another element or an intervening element can be present.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and the present invention, and will not be interpreted in an idealized or overly formal sense unless expressly so defined herein.

Meanwhile, in the following description of the present invention, a detailed description of known functions and configurations incorporated herein will be omitted when it may make the subject matter of the present invention unclear. The terms used in the specification are defined in consideration of functions used in the present invention, and can be changed according to the intent or conventionally used methods of clients, operators, and users. Accordingly, definitions of the terms should be understood on the basis of the entire description of the present specification.

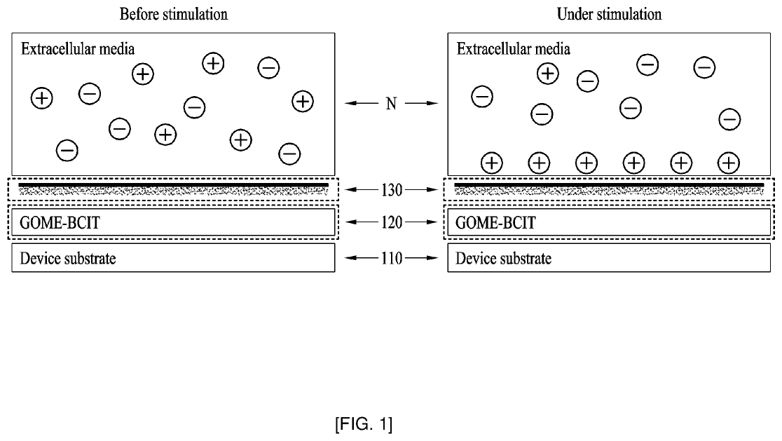

FIG. 1 illustrates a sectional view of a nerve stimulator electrode according to an embodiment of the present invention.

The nerve stimulator electrode according to an embodiment of the present invention includes a biocompatible solid electrolyte 120 and an ion diffusion barrier 130 formed on the biocompatible solid electrolyte 120, wherein the biocompatible solid electrolyte includes an ionic liquid and a biocompatible polymer matrix.

Therefore, in the nerve stimulator electrode according to an embodiment of the present invention, the biocompatible solid electrolyte 120 has mechanical properties similar to those of living tissue, so it does not cause an immune response or physical damage to living tissue when it comes in contact with the nerve N, and maintains a stable nerve N interface even when there is body movement. Accordingly, an electrical signal applied from a current signal generator can be delivered to the nerve N.

That is, the nerve stimulator electrode according to an embodiment of the present invention can deliver electrical stimulation without physical/chemical damage to the nerve N and living tissue, and especially it can be used as a digital treatment for incurable diseases where side effects and resistance are a concern due to continuous/long-term drug use and can be applied to regenerate damaged nerves.

The nerve stimulator electrode according to an embodiment of the present invention may include the biocompatible solid electrolyte 120.

According to an embodiment, the nerve stimulator electrode according to an embodiment of the present invention may include a device substrate 110 or a passivation layer, but these are not essential elements.

The device substrate 110 may be a flexible substrate.

In the nerve stimulator electrode according to an embodiment of the present invention, the biocompatible solid electrolyte 120 contains an ionic liquid and a biocompatible polymer matrix, so the biocompatible solid electrolyte 120 can move ions and form an electric double layer under an electric field.

Therefore, the nerve stimulator electrode according to an embodiment of the present invention may use the capacitive charge injection mechanism of the highly conductive biocompatible solid electrolyte 120 instead of the charge injection mechanism through oxidation/reduction reactions that can damage the nerve N.

For example, the biocompatible solid electrolyte 120 enables the movement of ions and the formation of an electric double layer under an electric field by uniformly distributing a biocompatible choline ionic liquid within a biocompatible polymer matrix and delivers electrical stimulation to nerves and biological tissues to generate an action potential.

More specifically, an electrode included in a pulse generator or a pulse generator can directly form an interface with the biocompatible solid electrolyte 120, and a voltage change generated from a pulse generator separates positive and negative ions from each other inside the biocompatible solid electrolyte 120, forming an electric double layer on different sides.

Here, since a potential difference is formed by an electric double layer at the interface between nerves and the biocompatible solid electrolyte 120, it can induce the formation of an electric double layer of ions that make up extracellular media ECM. More preferably, since a potential difference is formed by an electric double layer at the interface between nerves and the biocompatible solid electrolyte 120, it can induce the generation of ionic currents of ions that make up extracellular media ECM.

Therefore, since the nerve stimulator electrode according to an embodiment of the present invention uses this capacitive charge injection mechanism, there is no direct charge transfer unlike charge injection mechanisms based on electrochemical oxidation/reduction reactions, so there are no biological side effects.

In the nerve stimulator electrode according to an embodiment of the present invention, at least one of the mechanical modulus and tensile yield point of the biocompatible solid electrolyte 120 may be controlled depending upon the content of an ionic liquid included in the biocompatible solid electrolyte 120.

More specifically, the biocompatible polymer may have a glucosamine monomer, amines and hydroxyl groups capable of hydrogen bonding may be included in the monomer, multiple functional groups in the glucosamine monomer may induce strong hydrogen bonding, and mechanical properties such as high hardness may be provided by increasing the crystallinity of a biocompatible polymer.

When a choline-based biocompatible ionic liquid, which is an ionic liquid with biocompatibility, is introduced into a biocompatible polymer matrix, the hydrogen bonding force between polymer chains may be weakened, and the crystallinity of biocompatible polymers may be decreased with increasing content.

Therefore, since the high hardness of the polymer matrix weakens as the crystallinity of biocompatible polymer decreases, the nerve stimulator electrode according to an embodiment of the present invention may secure the flexibility of the biocompatible solid electrolyte 120 by reducing the mechanical modulus and increasing the tensile yield point.

The content of the ionic liquid included in the biocompatible solid electrolyte 120 may be 10 wt % to 50 wt %. When the content is less than 10 wt %, there is a problem in that the electrochemical properties of the material are not secured because the movement of ions and the formation of an electric double-layer are not implemented. When the content exceeds 50 wt %, there is a problem that a solid electrolyte cannot be formed because the hydrogen bonds between polymer chains are weakened by the ionic liquid.

Therefore, the nerve stimulator electrode according to an embodiment of the present invention may adjust the content of the ionic liquid to secure mechanical properties similar to the nerve N and biological tissue, thereby preventing physical damage when a nerve stimulator electrode is inserted into the body.

An ionic liquid with biocompatibility may be produced through a carboxylic anion substitution reaction with an anion which is the counter ion of a precursor containing choline cation.

Therefore, the ionic liquid may include a choline cation and a carboxylic anion pairing with the choline cation, and the choline cation may include at least one of choline bicarbonate, choline hydroxide and choline chloride, and the carboxylic anion may include at least one of acetate, propionate, glycolate, benzoate, tiglate, malate, succinate, tartrate, fumarate and maleate.

The carboxylic anion may be composed of a single species or a mixture of two or more carboxylic anion species in appropriate proportions.

In the nerve stimulator electrode according to an embodiment of the present invention, the mechanical properties of the biocompatible solid electrolyte may be controlled depending upon the crosslinking density of the biocompatible polymer included in the biocompatible solid electrolyte 120.

The mechanical properties may include, but are not limited to, at least one of swelling ratio, biodegradability, modulus, tensile yield point and free volume.

The biocompatible polymers may be biodegraded by hydration reactions caused by body fluids in the body environment and decomposition enzymes in body fluids and may have a low free volume within a biocompatible polymer matrix due to high hydrogen bonding strength.

Therefore, a swelling ratio and a biodegradation rate may be controlled by suppressing moisture content in a biocompatible polymer matrix by introducing a crosslinking agent and controlling crosslinking density when synthesizing a biocompatible polymer.

In addition, the biocompatible polymers may secure free volume through cross-linking by covalent bonds between chains and may provide flexibility for the nerve stimulator electrode according to an embodiment of the present invention by containing a high content of ions.

That is, in the nerve stimulator electrode according to an embodiment of the present invention, the mechanical properties such as a swelling ratio, biodegradability, modulus, tensile yield point, and free volume of the biocompatible polymer matrix may be controlled depending upon the crosslinking density of the biocompatible polymer included in the biocompatible solid electrolyte 120.

When the biocompatible polymer included in the biocompatible solid electrolyte 120 has a too low crosslinking density, there is a problem with the stability of the biocompatible solid electrolyte 120 material as it has a large swelling ratio in living and wet environments and thus shows a high biodegradation rate.

On the other hand, when the crosslinking density of the biocompatible polymer included in the biocompatible solid electrolyte 120 is too high, there is a problem of low flexibility due to high hardness due to an increase in modulus and a decrease in free volume, and there is a problem that the ionic conductivity of the solid electrolyte is reduced due to a decrease in the ion content in the biocompatible polymer matrix.

Therefore, the nerve stimulator electrode according to an embodiment of the present invention may adjust the crosslinking density of the biocompatible polymer to secure mechanical properties similar to the nerve N and biological tissue, thereby preventing physical damage when a nerve stimulator electrode is inserted into the body.

The biocompatible polymer may include at least one of chitosan, collagen, gelatin, fucoidan, alginate, hyaluronic acid, cellulose, polylactide, polyglycolide, polycaprolactone, polytrimethylenecarbolinecarbonate, polyaminoacid, polyorthoester, polyethylene oxide and a copolymer thereof. Preferably, the biocompatible polymer may be chitosan or a protein/polysaccharide biocompatible polymer matrix.

A nerve stimulator electrode material should have biocompatibility characteristics without immune rejection in the human body, and the stability of the material to maintain its function semi-permanently is also an important factor. Representative characteristics of biocompatible polymers include biocompatibility, biodegradability and mechanical properties. Therefore, the nerve stimulator electrode according to an embodiment of the present invention uses a biopolymer to secure the biocompatibility of the electrode material and control its biodegradability according to the purpose of use.

In addition, a biocompatible polymer matrix composed of a biopolymer and a choline-based ionic liquid may have mechanical properties similar to living tissue.

Therefore, the nerve stimulator electrode according to an embodiment of the present invention may be used in a nerve stimulator by applying a nerve stimulator electrode formed based on the biocompatible solid electrolyte 120 material, which is composed of a choline-based ionic liquid and a biocompatible polymer matrix and has low mechanical modulus, instead of a metal material-based electrode having high mechanical modulus.

The nerve stimulator electrode according to an embodiment of the present invention may include an ion diffusion barrier 130 to be formed on the biocompatible solid electrolyte 120.

The ion diffusion barrier 130 may prevent ion exchange and outflow between the biocompatible solid electrolyte 120 and body fluids, preventing damage to living tissue and preventing from reducing charge injection efficiency.

In addition, the nerve stimulator electrode according to an embodiment of the present invention may suppress the ion exchange reaction between body fluids and the electrode because the ion diffusion barrier 130 is formed on the surface of the biocompatible solid electrolyte 120, thereby being used for a long time by inserting it into the body.

The nerve stimulator electrode according to an embodiment of the present invention may include at least one of first and second interface functional groups formed by surface-modifying the ion diffusion barrier 130, thereby securing the adhesiveness and bioadhesiveness of the biocompatible solid electrolyte 120.

More specifically, the ion diffusion barrier 130 may include the first and second interface functional groups on the surface thereof, the first interface functional group may induce at least one of hydrogen and covalent bonds with the biocompatible solid electrolyte 120, and the second interface functional group may induce at least one of hydrogen and covalent bonds with the nerve N.

Therefore, the first interface functional group may be formed between the ion diffusion barrier 130 and the biocompatible solid electrolyte 120, and the second interface functional group may be formed between the ion diffusion barrier 130 and the nerve N.

For example, when graphene oxide is used as the ion diffusion barrier 130 and a chitosan polymer matrix is used as the biocompatible polymer matrix, adhesion properties between the graphene oxide ion diffusion barrier 130 and the biocompatible solid electrolyte 120 may be adjusted by functionalizing (3-glycidyloxypropyl)trimethoxysilane) or dopamine/polydopamine molecules on the surface of graphene oxide to induce covalent bonding with the primary amine group of the chitosan polymer matrix.

The first interface functional group may include at least one of 3-glycidyloxypropyl)trimethoxysilane (GPTMS), 3-glycidyloxypropyl)triethoxysilane (GPTES) and dopamine/polydopamine. According to an embodiment, the bridge alkyl chain of the first interface functional group may be controlled.

In addition, using hydrogen bonding through a carboxyl group present on the surface of graphene oxide material and covalent bonding through N-hydroxysuccinimide or dopamine/polydopamine functionalization, the adhesion properties between the graphene oxide ion diffusion barrier 130 and the nerve N/living tissue may be controlled and the interfacial stability therebetween may be improved.

The second interface functional group may include at least one of N-hydroxysuccinimide and dopamine/polydopamine.

In addition, the ion diffusion barrier 130 may be formed entirely or locally on the surface of the biocompatible solid electrolyte 120, and the ion diffusion barrier 130 may be physically bonded to the biocompatible solid electrolyte 120.

The nerve stimulator electrode according to an embodiment of the present invention may include a nano-micro pattern formed on the surface of the ion diffusion barrier 130.

The nano-micro pattern may be a nano-sized pattern, a micro-sized pattern, or a complex pattern of a nano-sized pattern and a micro-sized pattern.

The nano-micro pattern may have at least one shape of cylinder, hemisphere, cone, square pillar, square pyramid, triangular prism, triangular pyramid, hexagonal prism and hexagonal pyramid, and the cross-section may have at least one shape of circle, semicircle, square, triangle, hexagon and diamond.

Therefore, the ion diffusion barrier 130 uses a high aspect ratio and a nano-micro pattern to increase the effective area of an electric double layer between a nerve stimulator electrode composed of ions and the nerve N, enabling high charge injection capacity and charge injection efficiency.

The ion diffusion barrier 130 may be formed of a 2D material. For example, the ion diffusion barrier 130 may include at least one of graphene, graphene oxide, Mxenes, 2D transition metal carbides, 2D transition metal carbides nitrides, transition metal dichalcogenides (TMDCs), 2D metal organic frameworks (MOFs) and 2D covalent organic frameworks (COFs).

In addition, the nerve stimulator electrode according to an embodiment of the present invention uses the biocompatible solid electrolyte 120 unlike conventional metal and conductive polymer materials, thereby not involving an electrochemical reaction. Accordingly, it not only prevents pH changes and gas/bubble generation around the electrode, but also suppresses the ion exchange reaction between body fluids and the electrode by using the ion diffusion barrier 130, allowing it to be inserted into the body for a long time.

The nerve stimulator electrode according to an embodiment of the present invention is a biocompatible nerve stimulator electrode that constitutes a nerve interface of a bio-implantable neurostimulator and may be applied to a platform of electroceuticals that can treat various diseases by electrically stimulating the deep brain, motor cortex, responsive neurons, spinal cord, vagus nerve and the like.

The nerve stimulator electrode according to an embodiment of the present invention delivers electrical stimulation generated from a pulse generator to nerves and living tissue, thereby being used as a biocompatible nerve stimulator electrode.

In addition, the nerve stimulator electrode according to an embodiment of the present invention not only has electrical stimulation therapy and nerve regeneration functions, but also may be used as a medical device based on a feedback control system that monitors the user's body information and environment in real-time by integrating sensor technology with guaranteed biocompatibility and can even treat diseases through nerve stimulation.

That is, the nerve stimulator electrode according to an embodiment of the present invention is an electrode that can generate action potentials of nerve cells by effectively delivering electrical stimulation to the nerve surface and may be used in various fields such as ion conductors, electrodes for nerve stimulators, or bio-implantable devices.

FIG. 2 is a schematic diagram illustrating a method of manufacturing the nerve stimulator electrode according to an embodiment of the present invention.

The method of manufacturing the nerve stimulator electrode according to an embodiment of the present invention includes the same components as in the nerve stimulator electrode according to an embodiment of the present invention, so descriptions of the same components are omitted.

The method of manufacturing the nerve stimulator electrode according to an embodiment of the present invention includes a step (S110) of forming an ion diffusion barrier 130 on a substrate 101, a step (S120) of coating a biocompatible solid electrolyte solution 121 including an ionic liquid and a biocompatible polymer on the ion diffusion barrier 130, a step (S130) of curing the coated biocompatible solid electrolyte solution 121 and a step (S140) of separating the cured biocompatible solid electrolyte from the substrate.

First, the step (S110) of forming an ion diffusion barrier 130 on a substrate 101 of the method of manufacturing the nerve stimulator electrode according to an embodiment of the present invention is performed.

The method of manufacturing the nerve stimulator electrode according to an embodiment of the present invention includes forming the ion diffusion barrier 130, thereby preventing ion exchange between the biocompatible solid electrolyte 120 and body fluids in vivo and securing bioadhesive properties.

In the step (S110) of forming an ion diffusion barrier 130 on a substrate 101, a material for forming the ion diffusion barrier 130 may be coated on a substrate. The coating method may be at least one of dip coating, spray coating, spin coating and drop coating.

According to an embodiment, the step (S110) of forming an ion diffusion barrier 130 on a substrate 101 may further include a first surface treatment step of the ion diffusion barrier 130.

Preferably, the first surface treatment step may be performed to improve the adhesion efficiency of the interface between the ion diffusion barrier 130 and the biocompatible polymer electrolyte.

That is, the first surface treatment may be performed between the step (S110) of forming an ion diffusion barrier 130 on a substrate 101 and the step (S120) of coating a biocompatible solid electrolyte solution 121 including an ionic liquid and a biocompatible polymer on the ion diffusion barrier 130.

More specifically, in the method of manufacturing the nerve stimulator electrode according to an embodiment of the present invention, the ion diffusion barrier 130 may be coated on a substrate 111, and then the ion diffusion barrier 130 may be subjected to the first surface treatment, thereby coating and curing the solid electrolyte solution 121.

For example, the first surface treatment of the graphene oxide ion diffusion barrier 130 may be implemented by hydrolyzing and condensing (3-glycidyloxypropyl)trimethoxysilane on the graphene oxide surface.

Therefore, the first surface treatment may include at least one of silane functionalization and dopamine/polydopamine functionalization.

In addition, the dopamine/polydopamine functionalization may be applied to both the first interface functional group and the second interface functional group.

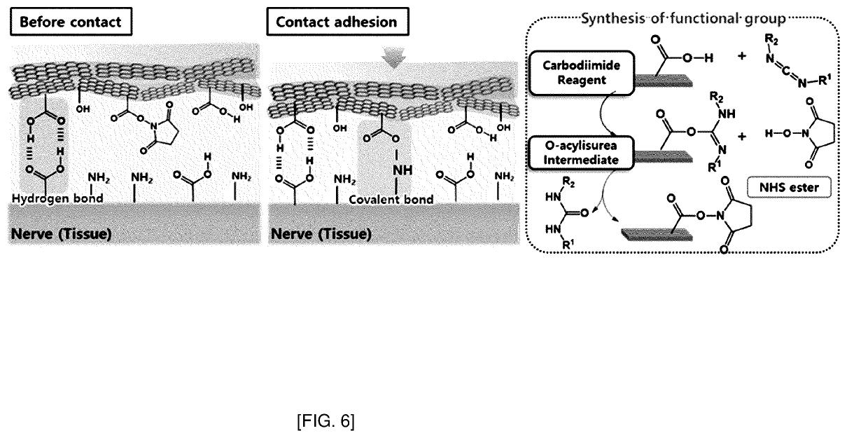

The first surface treatment technology will be described below with reference to FIG. 6.

Next, in the method of manufacturing the nerve stimulator electrode according to an embodiment of the present invention, the step (S120) of coating a biocompatible solid electrolyte solution 121 including an ionic liquid and a biocompatible polymer on the ion diffusion barrier 130 is performed.

The coating method may be at least one of dip coating, spray coating, spin coating and drop coating.

According to an embodiment, in the method of manufacturing the nerve stimulator electrode according to an embodiment of the present invention, an ionic liquid and biocompatible polymer may be synthesized before performing the step (S120) of coating a biocompatible solid electrolyte solution 121 including an ionic liquid and a biocompatible polymer on the ion diffusion barrier 130.

That is, the step (S120) of coating a biocompatible solid electrolyte solution 121 including an ionic liquid and a biocompatible polymer on the ion diffusion barrier 130 may further include a step of synthesizing an ionic liquid through anion substitution reaction.

For example, in the method of synthesizing a choline-based ionic liquid, a counter anion may be formed through anion substitution in choline hydroxide.

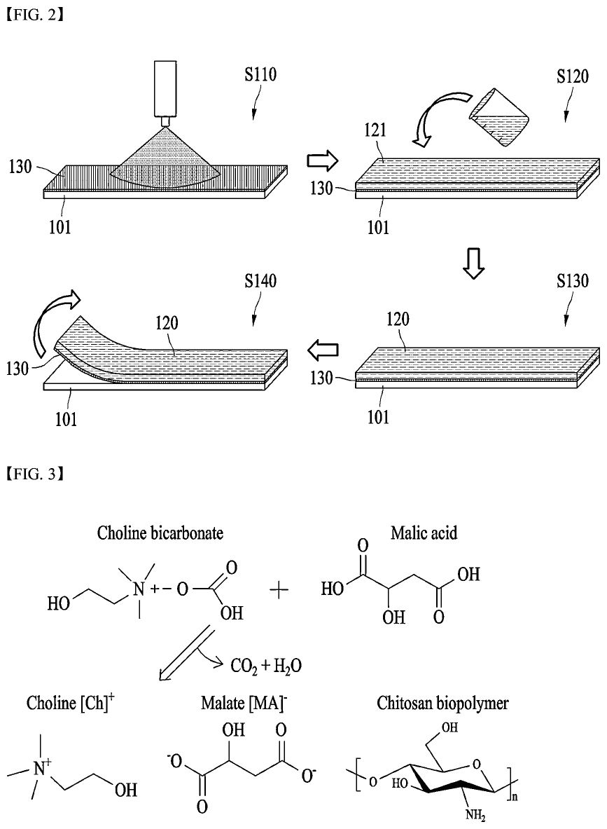

The step of synthesizing an ionic liquid through anion substitution reaction will be described in more detail below with reference to FIG. 3.

In addition, the step (S120) of coating a biocompatible solid electrolyte solution 121 including an ionic liquid and a biocompatible polymer on the ion diffusion barrier 130 may further include a step of synthesizing a biocompatible polymer using a cross-linking agent.

For example, a method of cross-linking a chitonic acid biocompatible polymer may induce ring opening reaction through a genipin-based cross-linking agent or may induce schiff base reaction through a polyethylene glycol and glutaraldehyde-based cross-linking agent.

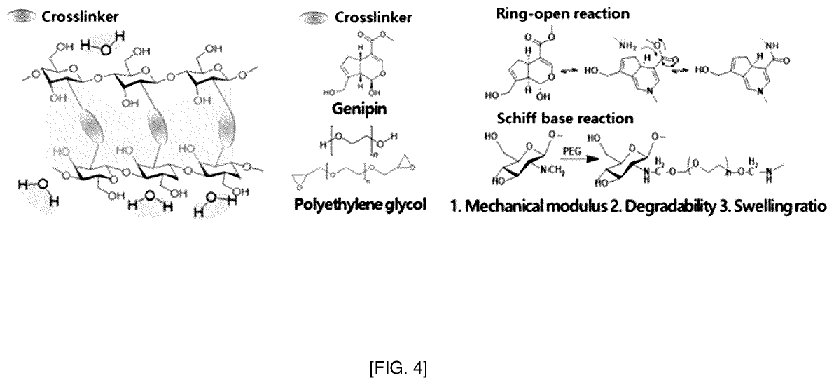

The step of synthesizing a biocompatible polymer using a cross-linking agent will be described in more detail with reference to FIG. 4.

At least one of the biocompatibility, swelling ratio, biodegradability and mechanical properties of the biocompatible solid electrolyte 120 may be controlled depending upon a cross-linking agent.

For example, in the case of glutaraldehyde that is a chitosan polymer cross-linking agent, it has high cross-linking efficiency through Schiff base reaction due to its high reactivity with a primary amine, but, when a cross-linking agent is added above a certain concentration, it exhibits cytotoxic properties.

On the other hand, in the case of genipin that is a biocompatible cross-linking agent extracted from plants, it has high biocompatibility even after cross-linking reaction.

In addition, the molecular weight of a cross-linking molecule may be controlled through the polymerization reaction of genipin, and the polymerizing cross-linking agent may control the mechanical properties such as modulus, free volume, and tensile yield point of the polymer matrix when cross-linking with the polymer chain.

Therefore, the method of manufacturing the nerve stimulator electrode according to an embodiment of the present invention may prevent physical damage when the nerve stimulator electrode is inserted into the living body by securing the mechanical properties of the nerve stimulator electrode according to an embodiment of the present invention to be similar to the nerves and the biological tissues depending upon a cross-linking agent.

In addition, by the method of manufacturing the nerve stimulator electrode according to an embodiment of the present invention, the biodegradability and mechanical properties of the nerve stimulator electrode according to an embodiment of the present invention may be adjusted depending upon crosslinking density and molecular weight.

The mechanical properties may include, but are not limited to, at least one of swelling ratio, biodegradability, modulus, tensile yield point and free volume.

The biocompatible polymers may be biodegraded by hydration reactions caused by body fluids in the body environment and decomposition enzymes in body fluids and may have a low free volume within a biocompatible polymer matrix due to high hydrogen bonding strength.

Therefore, a swelling ratio and a biodegradation rate may be controlled by suppressing moisture content in a biocompatible polymer matrix by introducing a crosslinking agent and controlling crosslinking density when synthesizing a biocompatible polymer.

In addition, the biocompatible polymers may secure free volume through cross-linking by covalent bonds between chains and may provide flexibility for the nerve stimulator electrode according to an embodiment of the present invention by containing a high content of ions.

That is, in the nerve stimulator electrode according to an embodiment of the present invention, the mechanical properties such as a swelling ratio, biodegradability, modulus, tensile yield point, and free volume of the biocompatible polymer matrix may be controlled depending upon the crosslinking density of the biocompatible polymer included in the biocompatible solid electrolyte 120.

When the biocompatible polymer included in the biocompatible solid electrolyte 120 has a too low crosslinking density, there is a problem with the stability of the biocompatible solid electrolyte 120 material as it has a large swelling ratio in living and wet environments and thus shows a high biodegradation rate.

On the other hand, when the crosslinking density of the biocompatible polymer included in the biocompatible solid electrolyte 120 is too high, there is a problem of low flexibility due to high hardness due to an increase in modulus and a decrease in free volume, and there is a problem that the ionic conductivity of the solid electrolyte is reduced due to a decrease in the ion content in the biocompatible polymer matrix.

The cross-linking agent may include at least one of genipin, polyethylene glycol, glutaraldehyde, transglutaminase, casein, gold nanoparticles, boron, polyhedral oligomeric silsesquioxane, iron, silver, titanium, dendrimer, albumin, silica, polyethylene glycol-block-polyproplyene glycol-block-poly ethylene glycol, carbodiimide compound, epoxide compound, alkyl halides, 2-chloro-dimethoxy-1,3,5-triazine, 2-chloro-methylpyridinium iodide, 1,1′-carbonyldiimidazole, diazomethane, divinyl sulfone, ethylenesulfide, glutaraldehyde, alkyl succinicanhydrides, acryl-chloride activated carboxylate, methacrylic anhydride, cyanogen bromide, adipic acid dihydrazide, hydrazine sulfate and derivatives thereof.

Next, in the method of manufacturing the nerve stimulator electrode according to an embodiment of the present invention, a step (S130) of curing the coated biocompatible solid electrolyte solution 121 is performed.

The curing may be performed using heat, UV, or high-energy radiation (electron beam, γ-ray). Preferably, a biocompatible solid electrolyte membrane 120 may be manufactured by directly irradiating UV to the coated biocompatible solid electrolyte solution 121.

Finally, in the method of manufacturing the nerve stimulator electrode according to an embodiment of the present invention, a step (S140) of separating the cured biocompatible solid electrolyte 120 from the substrate 101 is performed.

According to an embodiment, in the method of manufacturing the nerve stimulator electrode according to an embodiment of the present invention, a second surface treatment step of the ion diffusion barrier 130 may be performed after performing the step (S140) of separating the cured biocompatible solid electrolyte 120 from the substrate 101.

Therefore, the second surface treatment is formed on the ion diffusion barrier 130 surface in contact with the nerve, and may improve the adhesion efficiency of the interface between the ion diffusion barrier 130 and the nerve tissue.

For example, in the second surface treatment method of inducing covalent bonding between the graphene oxide ion diffusion barrier 130 and the nerve/living tissue, a carbodiimide reagent is used as a leaving group on the graphene oxide surface, and then O-acylisourea intermediate is reacted with N-Hydroxysuccinimide ester.

According to an embodiment, the second surface treatment may be performed through dopamine/polydopamine functionalization.

The second surface treatment technology will be described in more detail with reference to FIG. 6.

According to an embodiment, the method of manufacturing the nerve stimulator electrode according to an embodiment of the present invention may include a step (S110) of attaching the biocompatible solid electrolyte 120, separated from the substrate 101, onto the device substrate 110.

A nerve stimulator according to an embodiment of the present invention includes a pulse generator for generating electrical nerve stimulation pulses, the nerve stimulator electrode according to an embodiment of the present invention and a wire connection for electrically connecting the pulse generator and the nerve stimulator electrode.

The pulse generator included in the nerve stimulator according to an embodiment of the present invention is a circuit that determines the delivery form of pulses and generates voltage or current pulses of a desired pattern, and can generate a series of electrical nerve stimulation pulses.

The nerve stimulator electrode included in the nerve stimulator according to an embodiment of the present invention can deliver electrical nerve stimulation pulses received from the pulse generator to target nerve tissue as electrical stimulation.

The nerve stimulator electrode included in the nerve stimulator according to an embodiment of the present invention includes the same components as in the nerve stimulator electrode according to an embodiment of the present invention, so descriptions of the same components are omitted.

The respective ends of the wire connection included in the nerve stimulator according to an embodiment of the present invention are electrically connected to the nerve stimulator electrode according to an embodiment of the present invention and the pulse generator, so that electrical nerve stimulation pulses transmitted from the pulse generator may be delivered to target nerve tissue through the nerve stimulator electrode according to an embodiment of the present invention.

FIG. 3 illustrates a synthesis process of an ionic liquid used in the nerve stimulator electrode according to an embodiment of the present invention.

Referring to FIG. 3, by the method of synthesizing a choline-based ionic liquid, an ionic liquid of a choline cation and a malate anion may be formed through the condensation reaction of choline bicarbonate and malic acid.

For example, the ionic liquid used in the nerve stimulator electrode according to an embodiment of the present invention may be prepared as shown in FIG. 3.

More specifically, 13.41 g of malic acid was dissolved in 50 ml of methanol (Sigma-Aldrich) to make a 2 M malic acid solution, and then slowly added dropwise to a 0.8 M aqueous solution of choline bicarbonate made by mixing 35.30 ml of choline bicarbonate (Sigma-Aldrich) and 214.7 ml of water. Here, the molar ratio of malic acid:choline bicarbonate was 1:2.

The mixed solution was stirred at room temperature for 4 hours to remove carbon dioxide gas by an anion substitution reaction. Water and methanol, which were used as solvents in the mixed choline malate ([Ch]+[MA]−) solution, were removed by evaporating at 60° C. for 2 hours using a rotary evaporator.

To remove residual moisture in an ionic liquid, a pure choline malate ionic liquid may be obtained by drying in a vacuum oven at 60° C. for 48 hours.

FIG. 4 illustrates the cross-linking reaction of a biocompatible polymer used in the nerve stimulator electrode according to an embodiment of the invention.

For example, the biocompatible polymer used in the nerve stimulator electrode according to an embodiment of the invention may be prepared as shown in FIG. 4.

According to an embodiment, genipin solutions were prepared at different concentrations (0.5 mM, 1 mM, 2 mM, etc.), and a solvent may include an organic solvent such as ethanol, DMSO, or dimethylformamide.

The prepared genipin solution and chitosan solution were stirred at room temperature for different crosslinking times (0.5 h, 1 h, etc.). The mixed solution was poured into a glass petri dish and then dried at room temperature for 48 hours to obtain a genipin crosslinked chitosan film.

According to an embodiment, a small amount of formaldehyde (4 v/v % compared to added PEO) was added to the chitosan solution to form Schiff base, and then PEO, a cross-linking agent, was added at different concentrations (5, 10, 15, 20 wt compared to chitosan). The mixed solution stirred at room temperature for 24 hours is poured into a glass petri dish, and then dried at room temperature for 48 hours to obtain a PEO cross-linked chitosan film.

In addition, when synthesizing a biocompatible polymer used in the nerve stimulator electrode according to an embodiment of the invention, the mechanical properties, biodegradability or swelling ratio of the cross-linked biocompatible polymer may be controlled by adjusting at least one of the type and concentration of the biocompatible cross-linking agent.

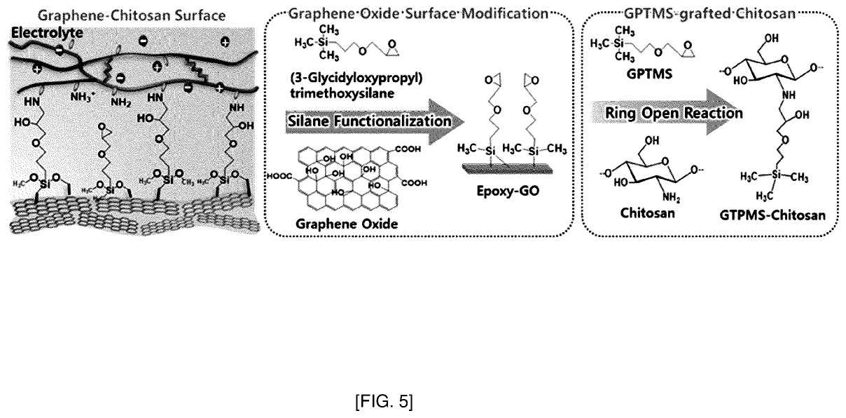

FIG. 5 illustrates a schematic diagram of a first surface treatment step of an ion diffusion barrier in the method of manufacturing the nerve stimulator electrode according to an embodiment of the present invention.

Referring to FIG. 5, surface treatment may be performed through silane functionalization using a methoxyl group on a graphene oxide surface.

Interface adhesion properties may be secured by inducing a covalent bond with the amine group of chitosan through a ring opening reaction of the epoxy group at the end of the surface-treated functional group.

For example, in the method of manufacturing the nerve stimulator electrode according to an embodiment of the present invention, the first surface treatment method of the ion diffusion barrier is to prepare 8 g to 10 g of aqueous graphene oxide solution (0.1 wt %˜0.5 wt %), perform sonication, and then spray-coat the aqueous graphene oxide solution onto a polytetrafluoroethylene (PTFE) substrate.

Next, (3-glycidyloxypropyl)trimethoxysilane (GPTMS) was prepared at a concentration of 0.5M in a toluene solvent, and then the graphene oxide surface was surface-treated by dipping at 70° C. for 48 hours and then rinsing with toluene.

FIG. 6 illustrates a schematic diagram of a second surface treatment step of an ion diffusion barrier in the method of manufacturing the nerve stimulator electrode according to an embodiment of the present invention.

Referring to FIG. 6, in the method of manufacturing the nerve stimulator electrode according to an embodiment of the present invention, hydrogen bonding between graphene oxide and a carboxyl group on the nerve surface can be induced by performing the second surface treatment step of the ion diffusion barrier.

For example, after preparing 4 ml of 2-(N-morpholino)ethanesulfonic acid) (MES) buffer 0.25M (pH=6.10), N-(3-Dimethylaminopropyl)-N′-ethylcarbodiimide hydrochloride) (EDC) may be dissolved at a concentration of 100 mM to the prepared MES buffer.

After dissolving N-hydroxysuccinimide (NHS) at a concentration of 50 mM in the prepared MES buffer and mixing, the second surface treatment was performed by dipping the graphene oxide surface coated on the substrate for 2 hours at room temperature.

### [Example 1]: Biocompatible Solid Electrolyte Containing 10 wt % of Ionic Liquid (BCIT-10 wt %)

0.2 g (2 wt %) of chitosan was mixed with 1 v/v % of acetic acid and stirred at room temperature for 24 hours to obtain a chitosan solution. 0.022 g (10 wt %) [Ch]⁺[MA]⁻ (choline malate), an ionic liquid (IL), was added to the chitosan solution dropwise and stirred for 24 hours.

The solution was sonicated to obtain a biocompatible electrolyte solution in which an ionic liquid was uniformly dispersed.

The solution was poured into a glass petri dish and dried at room temperature for 48 hours to obtain a biocompatible solid electrolyte.

### [Example 2]: Biocompatible Solid Electrolyte Containing 30 wt % of Ionic Liquid (BCIT-30 wt %)

A biocompatible solid electrolyte was prepared in the same manner as in Example 1 except that 0.086 g (30 wt %) of choline malate was contained.

### [Example 3]: Biocompatible Solid Electrolyte Containing 50 wt % of Ionic Liquid (BCIT-50 wt %)

A biocompatible solid electrolyte was prepared in the same manner as in Example 1 except that 0.2 g (50 wt %) of choline malate was contained.

### [Example 4]: Biocompatible Solid Electrolyte Containing Ion Diffusion Barrier and 10 wt % of Ionic Liquid (GOME-BCIT-10%)

A biocompatible solid electrolyte was prepared in the same manner as in Example 1 except that 0.029 g (10 wt %) of choline malate was contained compared to 0.2 g of chitosan.

### [Example 5]: Biocompatible Solid Electrolyte Containing Ion Diffusion Barrier and 30 Wt % of Ionic Liquid (GOME-BCIT-30%)

A biocompatible solid electrolyte was prepared in the same manner as in Example 1 except that 0.086 g (30 wt %) of choline malate was contained compared to 0.2 g of chitosan.

FIG. 7 is a graph illustrating the mechanical properties of the nerve stimulator electrode according to an embodiment of the present invention dependent upon the content of an ionic liquid.

Referring to FIG. 7, the nerve stimulator electrode according to an embodiment of the present invention shows that as the content of an ionic liquid increases, the content of ions that induce a plasticizing effect in the biocompatible polymer increases and the mechanical modulus decreases.

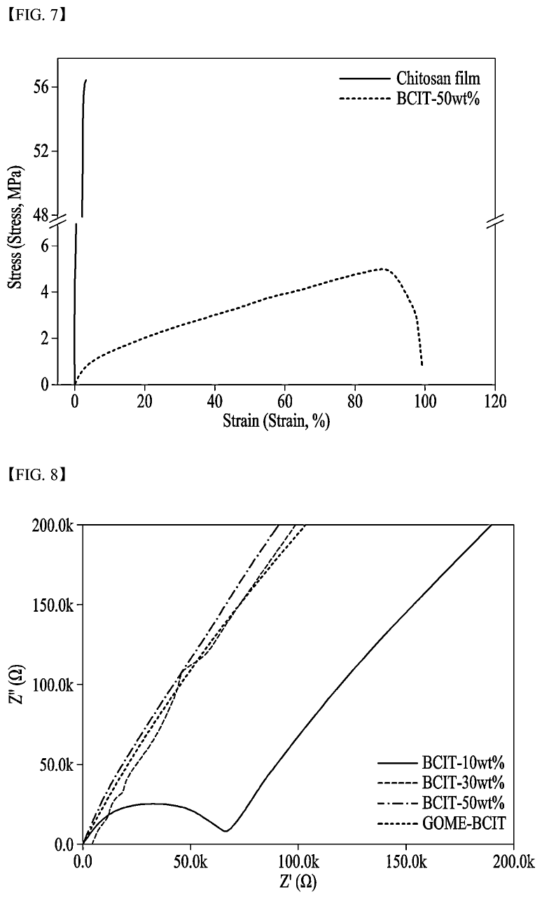

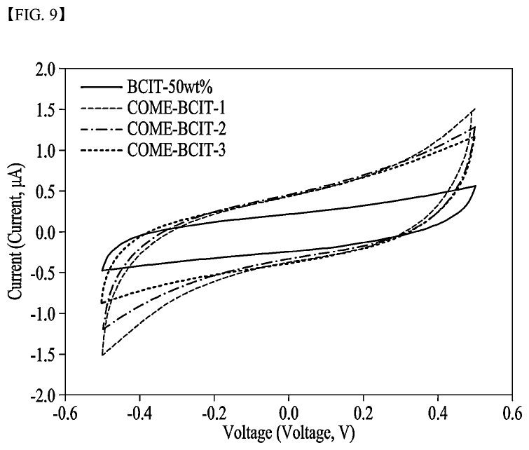

FIG. 8 is a graph illustrating changes in electrochemical properties of the nerve stimulator electrode according to an embodiment of the present invention dependent upon the content of an ionic liquid.

Referring to FIG. 8, Examples 1 to 3 (BCIT-10 wt %, BCIT-30 wt % and BCIT-50 wt %) of the nerve stimulator electrode according to an embodiment of the present invention show that as the content of an ionic liquid increases, ionic conductivity increases and impedance decreases.

In addition, it can be seen that the impedance is maintained even when graphene oxide, an ion diffusion barrier, is introduced to the biocompatible solid electrolyte surface (GOME-BCIT).

FIG. 9 illustrates a charge injection amount dependent upon the number of layers of an ion diffusion barrier of the nerve stimulator electrode according to an embodiment of the present invention.

Referring to FIG. 9, it can be seen that the high aspect ratio and nano/micro structure of graphene oxide used as an ion diffusion barrier increases the effective area of the electric double layer composed of ions, improving charge injection capacity, but, as the number of layers of the ion diffusion barrier increases from 1 to 3 layers (GOME-BCIT-1, GOME-BCIT-2, GOME-BCIT-3), the thickness of the graphene oxide increases and the capacitance decreases, reducing charge injection capacity.

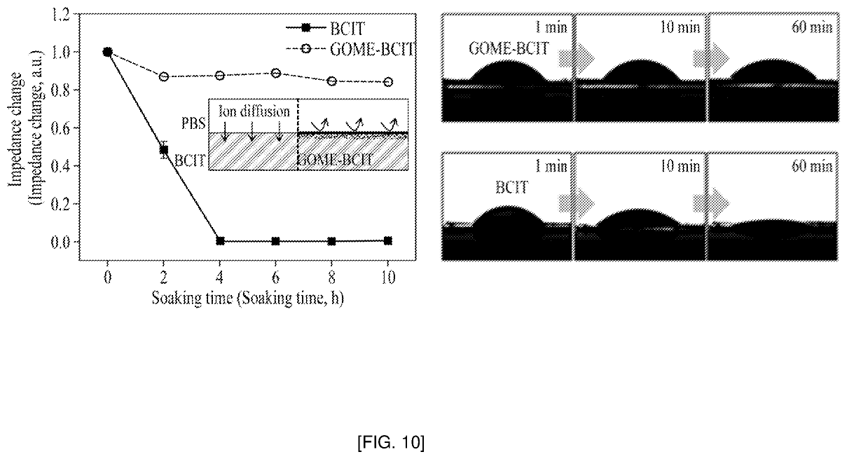

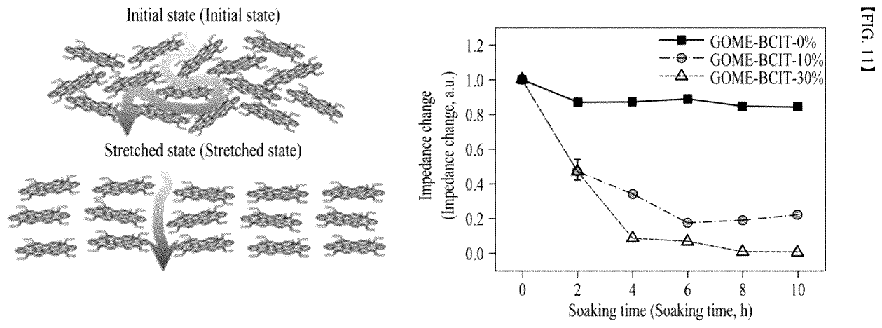

FIG. 10 illustrates a graph showing changes in electrochemical properties of the nerve stimulator electrode according to an embodiment of the present invention including an ion diffusion barrier and an image showing the transmittance within an electrode of a PBS buffer solution.

Referring to FIG. 10, it can be seen that, by using graphene oxide as an ion diffusion barrier (GOME-BCIT), outflow of ions within the nerve stimulator electrode material according to an embodiment of the present invention is prevented, thereby maintaining electrochemical properties.

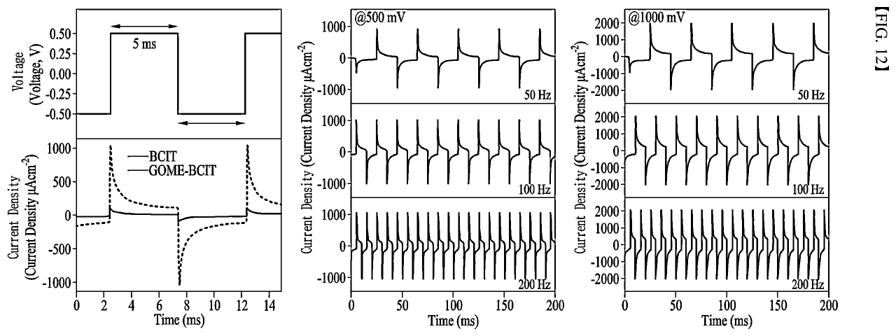

FIG. 11 illustrates an image and graph showing the ion outflow effect dependent upon the physical tension of the nerve stimulator electrode according to an embodiment of the present invention including an ion diffusion barrier.

Referring to FIG. 11, it can be seen that the electrochemical properties of the biocompatible solid electrolyte that contains an ion diffusion barrier and does not contain an ionic liquid (GOME-BCIT-0%) are not changed even when physical tensile strength increases, but, in the case of the biocompatible solid electrolyte (GOME-BCIT-10%) containing an ion diffusion barrier and 10 wt % of an ionic liquid and the biocompatible solid electrolyte (GOME-BCIT-30%) containing an ion diffusion barrier and 30 wt % of an ionic liquid, an ion movement path is formed within the graphene oxide layer, an ion diffusion barrier, as the physical tensile strength increases, so that the electrochemical properties thereof are changed due to ion outflow within the nerve stimulator electrode material according to an embodiment of the present invention.

FIG. 12 is a graph illustrating the charge injection efficiency of the nerve stimulator electrode according to an embodiment of the present invention.

Referring to FIG. 12, it can be seen that the charge injection efficiency of the nerve stimulator electrode according to an embodiment of the present invention changes depending upon the application duration, frequency, and voltage intensity.

Meanwhile, embodiments of the present invention disclosed in the present specification and drawings are only provided to help understanding of the present invention and the present invention is not limited to the embodiments. It will be apparent to those skilled in the art that various modifications can be made to the above-described exemplary embodiments of the present invention without departing from the spirit or scope of the invention.

1. A nerve stimulator electrode, comprising:
a biocompatible solid electrolyte; and
an ion diffusion barrier formed on the biocompatible solid electrolyte,
wherein the biocompatible solid electrolyte comprises an ionic liquid and a biocompatible polymer matrix.

2. The nerve stimulator electrode according to claim 1, wherein the biocompatible solid electrolyte allows ion movement and forms an electric double layer under an electric field.

3. The nerve stimulator electrode according to claim 1, wherein at least one of mechanical modulus and tensile yield point of the biocompatible solid electrolyte is controlled depending upon a content of the ionic liquid.

4. The nerve stimulator electrode according to claim 1, wherein mechanical properties of the biocompatible solid electrolyte is controlled depending upon a crosslinking density of the biocompatible polymer.

5. The nerve stimulator electrode according to claim 1, wherein the ionic liquid comprises a choline cation and a carboxylic anion paired with the choline cation,
wherein the choline cation comprises at least one of choline bicarbonate, choline hydroxide and choline chloride, and
the carboxylic anion comprises at least one of acetate, propionate, glycolate, benzoate, tiglate, malate, succinate, tartrate, fumarate and maleate.

6. The nerve stimulator electrode according to claim 1, wherein the biocompatible polymer comprises at least one of chitosan, collagen, gelatin, fucoidan, alginate, hyaluronic acid, cellulose, polylactide, polyglycolide, polycaprolactone, polytrimethylenecarbolinecarbonate, polyaminoacid, polyorthoester, polyethylene oxide and a copolymer thereof.

7. The nerve stimulator electrode according to claim 1, wherein the ion diffusion barrier comprises a first interface functional group and a second interface functional group on a surface thereof,
wherein the first interface functional group induces at least one of hydrogen and covalent bonds with the solid electrolyte, and
the second interface functional group induces at least one of hydrogen and covalent bonds with nerve.

8. The nerve stimulator electrode according to claim 1, wherein a nano-micro pattern is comprised on a surface of the ion diffusion barrier.

9. The nerve stimulator electrode according to claim 1, wherein the ion diffusion barrier comprises at least one of graphene, graphene oxide, Mxenes, 2D transition metal carbides, 2D transition metal carbides nitrides, transition metal dichalcogenides (TMDCs), 2D metal organic frameworks (MOFs) and 2D covalent organic frameworks (COFs).

10. A method of manufacturing a nerve stimulator electrode, the method comprising:
forming an ion diffusion barrier on a substrate;
coating a biocompatible solid electrolyte solution comprising an ionic liquid and a biocompatible polymer on the ion diffusion barrier;
curing the coated biocompatible solid electrolyte solution; and
separating the cured biocompatible solid electrolyte from the substrate.

11. The method according to claim 10, wherein the forming further comprises first surface-treating of the ion diffusion barrier.

12. The method according to claim 11, wherein the first surface-treating comprises at least one of silane functionalization and dopamine/polydopamine functionalization.

13. The method according to claim 10, wherein after the separating, second surface-treating of the ion diffusion barrier is performed.

14. A nerve stimulator, comprising:
a pulse generator for generating electrical nerve stimulation pulses;
the nerve stimulator electrode according to claim 1 that is in contact with a nerve; and
a wire connection for electrically connecting the pulse generator and the nerve stimulator electrode.
